(19) United States
(12) Patent Application Publication
(10) Pub. No.: US 2022/0062572 A1
(43) Pub. Date: Mar. 3, 2022

(54) A DEVICE FOR MAINTAINING AN AIRWAY IN A PATIENT

(71) Applicants: Kanag Baska, Brisbane (AU); Meenakshi Baska, Brisbane (AU)

(72) Inventor: Kanag Baska, Brisbane (AU)

(73) Assignee: Meenakshi Baska, Brisbane (AU)

(21) Appl. No.: 17/413,545

(22) PCT Filed: Dec. 13, 2019
(86) PCT No.: PCT/AU2019/051370

§ 371 Date: Jun. 12, 2021

(30) Foreign Application Priority Data
Dec. 13, 2018 (AU) 2018904736
Jan. 23, 2019 (AU) 2019900204

(51) Int. Cl. A61M 16/04; A61M 16/20; A61M 16/08

(52) U.S. Cl. CPC A61M 16/0445; A61M 16/0477; A61M 16/208; A61M 2210/1032; A61M 2202/0241; A61M 2202/0208; A61M 2205/3341; A61M 16/0833

(57) ABSTRACT

A device adapted for maintaining an airway in a patient, the device comprising a mask, the mask adapted to form a seal around the laryngeal inlet when properly inserted into a patient and an airway tube for providing ventilation gases and/or anaesthetic gases through the mask and to the lungs of the patient when the device is properly inserted in a patient, wherein the airway tube has a first proximal opening and a second proximal opening.

### TECHNICAL FIELD

The present invention relates to a laryngeal mask, more particularly, a laryngeal mask that also allows medical instruments, such as endotracheal tubes, to be inserted into a patient.

### BACKGROUND ART

Maintenance of a viable airway is critical to patient safety during surgical procedures conducted under general anaesthetic. Maintenance of a viable airway during such surgical procedures had, for many years, been achieved by insertion of an endo-tracheal tube into the patient. The endo-tracheal tube was typically inserted through the oral cavity or nasal cavity, into the larynx, through the vocal cord and into the trachea. As the endo-tracheal tube had to be inserted through the vocal cords, difficulty was often experienced in correctly positioning the endo-tracheal tube.

A number of supraglottic airway devices, or laryngeal masks, are now also available for use in maintaining a viable airway. The pioneering laryngeal mask was developed by Dr Archibald Brain and is described in British patent no. 2,111,394 (which corresponds to U.S. Pat. No. 4,509,514). The device is described as being an artificial airway device. The device comprises a curved, flexible tube opening at one end into the interior of a hollow mask portion shaped to conform to fit readily into the actual and potential space behind the larynx and to seal around the circumference of the laryngeal inlet without penetrating into the interior of the larynx. Commercial forms of this device have an inflatable and deflatable collar or cuff extending around the periphery of the mask. The inflatable collar is adapted to form the seal around the laryngeal inlet when the collar is inflated. Additionally, the mask portion included an inflatable posterior part which is adapted to press against the back of the throat and thereby increase the sealing pressure around the laryngeal inlet.

British patent no. 2,111,394 states that the shape and (when fitted) the inflatable part or parts of the mask ensure that it approximates closely to the shape of the space between the laryngeal inlet and the walls of the lower part of the throat behind it. Since the walls of tissue forming the back of the throat are relatively rigid, inflation of the mask forces it more tightly against the tissues surrounding the laryngeal inlet, so forming an airtight seal, while tending to anchor the mask in position.

In use of the device described in GB 2,111,394, the device is inserted through the mouth of the patient and down the throat past the epiglottis until the mask comes to rest with its distal end in the base of the throat, lying against the upper end of the normally closed oesophagus. The inflatable ring on the mask is then inflated to seal around the inlet to the larynx. The patent's airway in thus secure and unobstructed and the laryngeal mask can be connected directly to conventional anaesthetic circuit hosing for either positive pressure or spontaneously breathing.

A number of other suppliers are now also providing laryngeal masks. Indeed, the present inventor has developed a mask that is sold under the trade mark BASKA MASK. The present inventor has also obtained a number of patents for his products, including Australian patent numbers 2004260552, 2008291688 and 2010234212, as well as Australian patent application number 2015230697, the entire contents of which are incorporated herein by cross reference.

Supraglottic airway devices, which is another name for laryngeal masks, have found widespread use. They are used in emergency situations and in planned procedures. However, supraglottic airway devices may have some limitations during use as they may not achieve adequate ventilation nor adequate protection from lung aspiration. For these reasons, use of endotracheal tubes for intubation and ventilation of patients is still widely practised.

It is well known that insertion of an endotracheal tube can be very difficult at times and more so in an emergency situation. Endotracheal tubes can be difficult to properly place and can take an extended time to be properly inserted. In such circumstances, it may be significantly quicker to use a supraglottic airway device to facilitate the establishment viable airway in a patient.

If a supraglottic airway device has been inserted into a patient but there is concern that it is not achieving adequate ventilation or achieving adequate protection of the lungs against aspiration, an endotracheal tube may then be inserted. Insertion of the endotracheal tube in those circumstances is warranted to isolate and protect the airway and more importantly, to achieve adequate oxygenation to overcome any risk of hypoxia, brain damage or death.

In circumstances where a supraglottic airway device has been inserted into the patient, it may be possible to insert an endotracheal tube into the patient through the supraglottic airway device. It is important to maintain delivery of oxygen to the patient without interruption during the process of intubation of an endotracheal tube through a supraglottic airway device. Time is of the essence during insertion of the endotracheal tube as hypoxia can cause brain damage in a very short time. Intubation of an endotracheal tube through a supraglottic airway device is not easy and it can take considerable time, especially in the hands of an inexperienced operator. Bronchoscopes can be useful in guiding an endotracheal tube through the supraglottic airway device, but these may not be available in some settings. Even if available, it might take considerable time to set up such equipment, especially in an unexpected emergency situation.

During the process of inserting an endotracheal tube and/or a bronchoscope through a conventional supraglottic airway device/ laryngeal mask, the patient cannot be ventilated to be oxygenated and/or anaesthetised, which compromises the safety of the patient. Further, with currently available laryngeal masks, an endotracheal tube having a maximum inner diameter of about 7.5 mm is about the largest endotracheal tube that can be used.

It will be clearly understood that, if a prior art publication is referred to herein, this reference does not constitute an admission that the publication forms part of the common general knowledge in the art in Australia or in any other country.

### SUMMARY OF INVENTION

The present invention is directed to a laryngeal mask, which may at least partially overcome at least one of the abovementioned disadvantages or provide the consumer with a useful or commercial choice.

With the foregoing in view, the present invention in one form, resides broadly in a device adapted for maintaining an airway in a patient, the device comprising a mask, the mask adapted to form a seal around the laryngeal inlet when properly inserted into a patient and an airway tube for providing ventilation gases and/or anaesthetic gases through the mask and to the lungs of the patient when the device is properly inserted in a patient, wherein the airway tube has a first proximal opening and a second proximal opening.

In one embodiment of the present invention, the airway tube comprises a bifurcated tube having a first proximal opening and a second proximal opening. In one embodiment, the airway tube comprises a first tube having a second tube extending from a side thereof.

In one embodiment, the airway tube comprises a bifurcated tube. In one embodiment, the airway tube has a distal portion and the airway tube comprises a bifurcated tube forming a proximal portion of the airway tube.

Throughout this specification the term “proximal” will refer to a portion that is located closer to the surgeon or operator during use and the term “distal” will refer to a portion that is located further away from the surgeon or operator during use.

In one embodiment, the bifurcated portion may be formed as a separate portion that is attached to a distal portion of the airway tube. The airway tube or the bifurcated portion (or both) may be provided with one or more connectors to enable the distal portion to be connected to the bifurcated portion.

In other embodiments, the bifurcation may be located closer to the distal end of the airway tube.

The airway tube may comprise a stem that connects to or is formed with the mask at a distal end thereof. The stem may comprise a single tube, or a unitary tube, or a plurality of tube portions connected together. The airway tube may comprise a first tube and a second tube to form suction and vent channels in the device.

In one embodiment, the airway tube comprises a stem, a first airway tube and a second airway tube, the first airway tube and the second airway tube being in fluid communication with the stem.

In one embodiment, the first opening is connected to a source of ventilating gas and/or anaesthetic gas during use and the second opening provides access to the airway tube to enable an endotracheal tube to be inserted through the airway tube and into the larynx of a patient.

In one embodiment, at least one of the first opening or the second opening may be provided with a valve, a seal or a non-return valve. In one embodiment, both the first opening and the second opening are provided with a valve, a seal or a non-return valve. In another embodiment, a valve, a seal or a non-return valve is located upstream of a bifurcation point in at least one of the tubes located upstream of the bifurcated point.

In another embodiment where the airway tube has a first tube and a second tube being in fluid communication with an airway tube stem, a valve, a seal or a non-return valve may be located in the first tube, or in the second tube, or a valve, a seal or non-return valve may be located in each of the first tube and the second tube.

The non-return valve may allow for passage of gases from a proximal end to a distal end of the device, but may prevent exit of gases from a proximal end of the device. The seal can also prevent exit of gases from a proximal end of the device.

In another embodiment, a valve, a seal or a non-return valve may be located in the stem of the airway tube.

In one embodiment, a valve, a seal or a non-return valve is located at or near the first opening and the first opening is adapted to have an endotracheal tube or other instrument passed therethrough.

In another embodiment, the airway tube comprises an airway stem tube and a bifurcated connector is attached to the proximal end of the airway tube stem. The bifurcated connector has a first arm and a second arm. A valve, a seal or a non-return valve may be located in the first arm, or in the second arm, or a valve, a seal or non-return valve may be located in each of the first arm and the second arm.

In one embodiment, the non-return valve may comprise a duckbill valve or a slit valve. In one embodiment, the non-return valve comprises one or more valve portions, each valve portion having a surface extending inwardly from a periphery thereof, the valve portion having one edge that, in use, abuts an edge of an inwardly extending surface of an adjacent valve portion or a side wall of the airway tube and another edge that, in use, abuts an edge of an inwardly extending surface of an adjacent valve portion or a side wall of the airway tube when in the closed position, the surface having a skirt extending therefrom. In one embodiment, the surface and the skirt define a closed upstream region and an open downstream region. In use, the edges of the surface and edges of the skirts of one valve portion abut on the respective edges of the surface and edges of the skirt of an adjacent valve portion when in the closed position or abut on or a side wall of the airway tube. In this manner, a seal is formed. Further, if gas tries to flow from the downstream end to the upstream end, the gas will flow into the open downstream region and act to push the valve portions into contact with each other.

In one embodiment, the valve comprises three valve portions. In another embodiment, the valve comprises four valve portions. The valve may comprise more than four valve portions. Although the valve may also comprise two valve portions, it is believed that a more effective seal will be obtained when three or more valve portions are used. The valve may also comprise a single valve portion that seals against the side wall or walls of the tube in which it is mounted or located.

When an endotracheal tube is inserted through the valve, the surfaces and skirts of the valve portions will surround the endotracheal tube and provide a seal over an extended length of the endotracheal tube, thereby improving the seal around the endotracheal tube. If gas tries to flow from the downstream end to the upstream and, the gas will also enter the open downstream ends of the valve portions and force the valve portions into closer contact with the endotracheal tube, thereby improving the seal.

The mask may comprise any mask that is capable of forming a seal around the laryngeal inlet of the patient. In one embodiment, the mask may comprise an inflatable cuff that can be selectively inflated and deflated through an inflation tube. In this embodiment, the mask may be similar to the mask of airway devices sold by the Laryngeal Mask Company and invented by Dr Archibald Brain. An example of such a mask is described in U.S. Pat. No. 4,509,514.

In other embodiments, the mask portion may comprise a resilient conformal proportion that can form a seal around the laryngeal opening without requiring inflation. Some examples of these types of masks are given in Australian patent/patent application numbers 2004260552, 2008291688 and 2010234212, and 2015230697 in the name of the present applicants.

In one embodiment, the airway tube comprises a primary airway tube having a secondary airway tube or airway passage joining or entering into the primary airway tube.

In a second aspect, the present invention provides a bifurcated connector for attachment to a proximal end of an airway tube stem of a laryngeal mask, the bifurcated connector having a first arm and a second arm, a valve, a seal or a non-return valve being located in the first arm, or in the second arm, or a valve, a seal or non-return valve being located in each of the first arm and the second arm.

In a third aspect, the present invention provides a method for positioning endotracheal tube into a patient, the method comprising establishing an airway in the patient using the device of the first aspect of the present invention, providing ventilation gas and/or anaesthetic gas to the patient through one of the first opening or second opening of the device, and inserting an endotracheal tube through the other of the first opening on the second opening whilst maintaining supply of the ventilation gas and/or the anaesthetic gas.

In a fourth aspect, the present invention provides a device adapted for maintaining an airway in a patient, the device comprising an airway tube for providing ventilation gases and/or anaesthetic gases to the lungs of the patient when the device is properly inserted in a patient, wherein the airway tube has a valve, a seal or a non-return valve, or the device is fitted with a connector having a valve, a seal or a non-return valve.

The device of the fourth aspect of the present invention may comprise an endotracheal tube. The device of the fourth aspect of the present invention may comprise a supraglottic airway device. The device may comprise a mask, the mask adapted to form a seal around the laryngeal inlet when properly inserted into a patient.

In one embodiment, the valve, seal or non-return valve is fitted to the airway tube of the device. In one embodiment, the valve, seal or non-return valve is fitted to the proximal end of the airway tube of the device. In one embodiment, the valve, seal or non-return valve is located in a connector that is attached to a proximal end of the airway tube of the device.

In order to use an embodiment of the device in accordance with the fourth aspect of the present invention that has a mask such that the device of the fourth aspect of the present invention can be used as a conventional supraglottic airway device, a connector or a short tube may be inserted through the valve, seal or non-return valve to open the valve, seal or non-return valve. Ventilation gases and/or anaesthetic gases may then be provided in a conventional manner through the connector or short tube.

In some embodiments, an endotracheal tube may be inserted through the valve, seal or non-return valve.

In some embodiments, a connector or adapter having a first proximal opening and the second proximal opening, at least one of the first or second proximal openings having a valve, seal or non-return valve therein and a distal end is connected to the airway tube such that the distal and extends through the valve, seal or non-return valve of the device. Ventilation gases and/or anaesthetic gases may be connected to one of the first or second proximal openings, especially to a proximal opening that has no valve, seal or non-return valve, and a bronchoscope or other surgical instrument may be inserted through the other proximal opening which has a valve, seal or non-return valve.

In one embodiment, the airway tube of the device has a relatively large diameter and the valve, seal or non-return valve is fitted into the relatively large diameter airway tube. A connector or a short tube having a different diameter, especially a smaller diameter, can be inserted through the valve, seal or non-return valve of the airway tube of the device, to effectively provide a simple adapter that allows smaller diameter fittings to be connected thereto.

In embodiments of the fourth aspect of the present invention where the device is an endotracheal tube, other instruments, such as a bronchoscope, may be inserted through the valve.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 shows a view of a conventional airway device known from the prior art;

FIG. 2 shows a view of device in accordance with one embodiment of the present invention;

FIG. 3 shows a view of a device in accordance with another embodiment of the present invention;

FIG. 4 shows a view of a device in accordance with a further embodiment of the present invention;

FIG. 5 shows a view of a device in accordance with another aspect of the present invention;

FIG. 6 shows a view of the device shown in FIG. 5 without a bifurcated adaptor;

FIG. 7 shows a top view of a one-way valve suitable for use in an embodiment of the present invention;

FIG. 8 shows a bottom view of the one way valve shown in FIG. 7;

FIG. 9 shows a top isometric view of the one way valve shown in FIG. 7 ready for final assembly into the form shown in FIG. 7;

FIG. 10 is a side isometric view of the one way valve ready for assembly;

FIG. 11 is a front view of FIG. 10;

FIG. 12 is a side sectional view taken along section A-A in FIG. 11; and

FIG. 13 is a side sectional view taken along section B-B in FIG. 11.

### DESCRIPTION OF EMBODIMENTS

Those skilled in the art will appreciate that the drawings have been provided for the purposes of illustrating preferred embodiments of the present invention. Therefore, it will be understood that the present invention should not be considered to be limited solely to the features as shown in the attached drawings.

FIG. 1 shows a prior art device for establishing an airway in a patient in the form of a laryngeal mask that is currently available for purchase. The laryngeal mask 10 shown in FIG. 1 comprises an airway tube 12 and a mask portion 14. The mask portion 14 includes a backing plate 16 and an inflatable cuff 18 attached to the backing plate 16. An inflation tube 20 having a connector 22 that enables the inflation tube 20 to be connected to a syringe is in fluid communication with the inflatable cuff 18. The airway tube 12 is connected to the mask portion 14 by a connector 24. In some embodiments, the airway tube 12 may be integrally formed with the mask portion 14. A connector 26 located at a proximal end of the tube 12 enables the tube 12 to be connected to a tube that can supply ventilation gases or anaesthetic gases.

In use of the device 10 shown in FIG. 1, the cuff 18 is deflated and the mask is inserted into the throat of the patient. When the mask is properly located in the laryngo-pharynx, the cuff 18 is inflated so that a seal is formed around the laryngeal inlet of the patient. A gas supply tube is connected to connector 26 so that gas is supplied down airway tube 12 and into a chamber defined by backing plate 16 and inflatable cuff 18 and thereafter into the lungs of the patient.

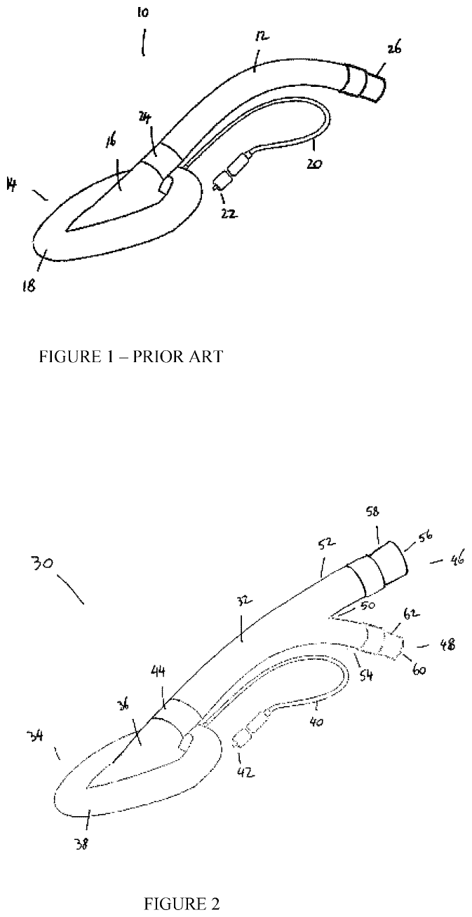

FIG. 2 shows a view of a device for maintaining an airway in a patient in accordance with an embodiment of the present invention. The device 30 shown in FIG. 2 has a number of features that are similar to that shown in FIG. 1. For example, the device has an airway tube 32 and a mask portion 34. The mask portion 34 includes a backing plate 36 and an inflatable cuff 38 that is inflated via inflation tube 40. Inflation tube 40 is connected to a syringe by a connector 42. This also allows the inflated cuff to be selectively deflated, such as for removal of the device from the patient following completion of a procedure. A connector 44 connects the airway tube 32 to the mask portion 34. Alternatively, the mask portion 34 may be integrally formed with the airway tube 32.

Airway tube 32 comprises a bifurcated airway tube that has a first proximal end generally referred to at 46 and a second proximal end generally referred to at 48. A bifurcation point 50 is formed in the airway tube. The airway tube may be considered as having a main airway tube 52 and a side tube 54. Airway tube 52 has a first opening 56 and a non-return valve or a duckbill valve 58. Second airway tube 54 has a second opening 60 which is formed by a connector 62 that is attached to the distal end of the airway tube 54.

In use of the device 30 shown in FIG. 2, the device is inserted into the patient with the inflatable cuff 38 being in a deflated condition. When properly inserted, the inflatable cuff is inflated form a seal around the laryngeal inlet. The connector 62 of the second airway tube 54 is attached to a source of ventilation gas or anaesthetic gas so that ventilation or anaesthetic gases can be supplied to the patient. If no devices are connected to the second airway tube 52, the non-return valve or duckbill valve 58 prevents gas from leaving through opening 56 of the second airway tube 52. This maintains the requisite pressure within the device to ensure adequate ventilation to the patient.

If it is desired to insert an endotracheal tube into the patient through the device 30, the endotracheal tube (not shown) can be inserted through non-return valve or duckbill valve 58 and along the airway tube 52 and the main airway tube 32, through the airway chamber defined by the backing plate 36 and the inflatable cuff 38 and into the laryngeal opening of the patient. Whilst the endotracheal tube is being inserted into the patient, ventilation gases or anaesthetic gases can continue to be supplied to the patient through second airway tube 54. Accordingly, ventilation of the patient is not interrupted during insertion of the endotracheal tube. This has not previously been possible when inserting an endotracheal tube into a patient through a laryngeal mask. The non-return valve or duckbill valve 58 can also form a seal around the endotracheal tube to thereby ensure that pressure within the airway device 30 is maintained during insertion of the endotracheal tube into the patient.

The non-return valve 58 may comprise a duckbill valve, which will be well known to person skilled in the art. A number of other non-return valves are also known for use in anaesthesia and any of those valves may also be used.

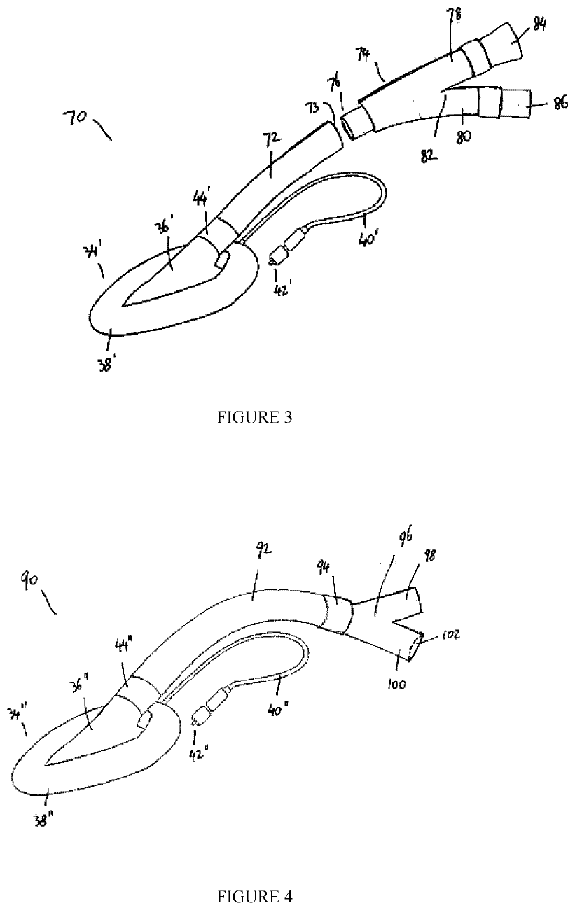

FIG. 3 shows a perspective view of a device 70 in accordance with a further embodiment of the present invention. The device 70 has a number of features in common with device 30 shown in FIG. 2 and for convenience, those features would be denoted by similar reference but with the addition of a ′ thereto. These features need not be described further.

The device 70 shown in FIG. 3 has an airway stem 72 connected via connector 44′ to the mask portion 34′. The airway stem 72 is in the form of a distal airway tube. A bifurcated tube 74 having a connector 76 is connected to the proximal end 73 of distal airway stem 72. The bifurcated airway tube 74 has a first tube 78 and a second tube 80. The tubes 78, 80 extend from a bifurcation point 82. The first tube 78 has a non-return valve or duckbill valve 84. The second airway tube 80 has an airway connector 86.

In use, the bifurcated airway tube 74 is connected to the airway tube stem 72 and the device 70 is inserted into the patient as described above with reference to the device of FIG. 2. The connector 86 is connected to a source of ventilation gases or anaesthetic gases. If it is desired to insert an endotracheal tube to the patient through the airway tube 70, the endotracheal tube may be inserted through non-return valve or duckbill valve 84 and second airway tube 78. Other surgical instruments, such as bronchoscopes, endoscopes, endoscopic lights, etc, may also be inserted through second airway tube 78. Ventilation of the patient can continue during insertion of the endotracheal tube or other instruments.

FIG. 4 shows a further embodiment of the present invention. The device 90 shown in FIG. 4 has a number of features in common with device 30 shown in FIG. 2 and for convenience, those features would be denoted by similar reference but with the addition of ″ thereto. These features need not be described further.

The device 90 shown in FIG. 4 has a main airway tube 92 that is in the form of a single unitary tube connected to or formed with the mask portion 34. A connector 94 is affixed to the proximal end of the airway tube 92. A bifurcated connector 96 is, in turn, connected to the connector 94. This places the bifurcated connector 96 into fluid communication with the airway tube 92. The bifurcated connector 96 has a first arm 98 and a second arm 100. A non-return valve or duckbill valve shown schematically at 102 is fitted to second arm 100.

In use of the device 90 shown in FIG. 4, the device is inserted into the patient as described with reference to the device of FIG. 2. The bifurcated connector 96 may be connected to connector 94 before or after insertion of the device 90 into the patient. Once connected, a gas supply tube is fixed to second arm 98 of bifurcated connector 96. This enables supply of ventilation gases or anaesthetic gases to the patient. If it is desired to insert an endotracheal tube through the device 90 to the patient, it may be inserted through second arm 100 of bifurcated connector 96. Again, supply of ventilation gases to the patient may be continued through first arm 98 during insertion of the endotracheal tube.

Devices in accordance with preferred embodiments of the present invention enable an airway to be established using the particular devices. Due to the other arm of the bifurcated part of the device having a non-return valve, a seal or a valve therein, the pressurised ventilation gases cannot escape through the other arm of the bifurcated part of the device. This enables viable ventilation to be continued. If an endotracheal tube is inserted through the other arm or tube of the bifurcated part of the device, a seal is formed around the endotracheal tube, which again prevents ventilation gases from leaving the device through the other arm or tube of the bifurcated part of the device. Thus, ventilation can continue during insertion of the endotracheal tube.

A further advantage of devices in accordance with preferred embodiments of the present invention is that larger diameter endotracheal tubes can be used in conjunction therewith. For example, in the embodiment shown in FIGS. 2 to 4, the main airway tube 32, 72, 92 is formed as a unitary tube of relatively large inner diameter. Accordingly, an endotracheal tube having an outer diameter that is almost as large as the inner diameter of the airway tube can be inserted into the patient without blocking supply of ventilation gases to the patient.

In alternative embodiments, the non-return valves shown in FIGS. 2, 3 and 4 may be replaced by manually operable valves or may be replaced by seals or closures. If a seal or closure is provided, when an endotracheal tube is inserted through the seal or closure, the seal closure forms a seal around the endotracheal tube to thereby prevent escape of ventilation gases from the other opening of the bifurcated part of the device. Similarly, if a manually operated valve is used, the manually operated valve may be opened when it is desired to insert an endotracheal tube therethrough. Desirably, the manually operated valve forms a seal around the outside of the endotracheal tube. Additionally, a removable cap may be placed over the opening to the valve. Indeed, removable caps may be provided to one or more or even all of the openings of the devices in accordance with the present invention.

Other medical equipment besides or as well as endotracheal tubes may also be inserted through the device of the present invention. For example, other equipment such as bronchoscopes, guide wires, ultrasound equipment, endoscopic lights, endoscopes, clamps, forceps, etc may also be inserted.

Preferred forms of the present invention are provided with a valve, a seal or a non-return valve in the arm that receives the endotracheal tube (ETT) or other instrument(s). The valve, seal or non-return valve prevent gas from passing out of the device therethrough when there is no ETT or other instrument present. Similarly, the valve, seal or non-return valve also form a seal around the ETT or other instrument when the ETT or other instrument is inserted therethrough and also prevents gas from passing out of the device therethrough when the ETT or other instrument is present.

Intubation with an ETT through supraglottic airway devices (SGADs) is presently attempted and done mainly in a non-breathing patient. Maintenance of positive pressure in the Airway is an absolute necessity to keep the lung expanded to avoid collapse of the lung in a non-breathing patient. Therefore IPPV (intermittent positive pressure ventilation) with or without CPAP (continuous positive airway pressure) is absolutely essential in an already compromised patient to maintain lung ventilation, lung expansion, oxygenation, and prevent hypercarbia.

An attempt to insert an ETT or any other device through any of the other existing SGADs leaves the airway open to the atmospheric pressure and to the atmospheric air for a considerable time compromising oxygenation and collapse of the lungs causing hypoxia and possible hypercarbia as this process may take time.

The second airway tube in the present invention enables ippv, cpap, oxygenation & lung expansion with the help of the non-return valve, the valve or the seal, which enables passage of ETT or other equipment whilst retaining the airway pressure maintaining lung expansion and oxygenation to the patient that is what is eventually to be achieved by intubating the patent with an ETT.

All other SGADS currently known to the inventor precisely cannot do this without a main airway tube to ventilate and the valve, seal or non-return valve on the secondary airway tube to maintain the airway pressure. This valve, seal or non-return valve enables effectively to stop the air leak around the instrument that is being passed through it sustaining the airway pressure.

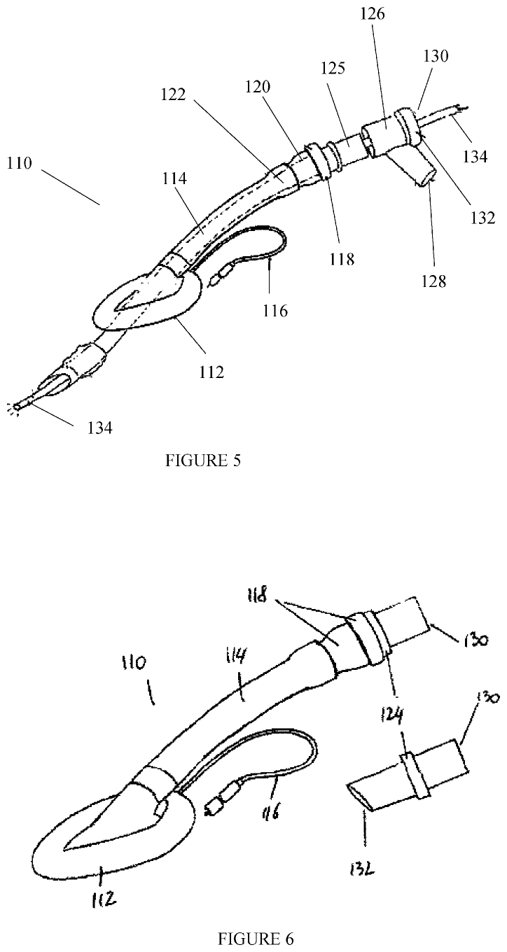

FIG. 5 shows a view of an airway device in accordance with an embodiment of the fourth aspect of the present invention. The airway device 110 comprises a mask portion 112 and an airway tube 114. Inflation tube 116 can be used to selectively inflate and deflate the mask portion 112. The airway tube 114 is of a larger diameter than in conventional supraglottic airway devices.

A valve 118, which is suitably a non-return valve or a one-way valve, such as a duck bill valve, is fitted to the proximal end of the airway tube 114. The valve 118 may be directly mounted into the proximal end of the airway tube 114, or it may form part of an adapter 120 that is connected to the proximal end of airway tube 114 (as shown in FIG. 5).

If the device 110 shown in FIG. 5 is to be used as a supraglottic airway device, a tube 122 can be inserted through the valve 118. This opens the valve. A connector 125 at the end of the tube can then be connected directly to an airway connector for ventilation gases and/or anaesthetic gases. This is not shown in FIG. 5. Rather, FIG. 5 shows a bifurcated adapter 126 being connected to the connector 125. Bifurcated adapter 126 has a first proximal end 128 that can be connected to a tube supplying ventilation gases and/or anaesthetic gases. The adapter 126 has a second proximal end 130 that is fitted with a valve, seal or non-return valve, such as a duck bill valve, 132. This enables a bronchoscope or other surgical instrument to be inserted through the valve 132. In this arrangement, the device shown in FIG. 5 operates in a manner that is similar to the device as shown in FIGS. 1 to 4.

The tube 122 shown in FIG. 5 may be just a conventional surgical tube for supplying gas, or it may comprise the tube of an endotracheal tube. FIG. 5 shows the tube 122 being in the form of an endotracheal tube. FIG. 5 shows the in-use arrangement of the bronchoscope and endotracheal tube relative to the airway device 110.

FIG. 6 shows the device of FIG. 5 without the bifurcated adaptor 126 being connected thereto. In FIG. 6, a connector 124 has a standard connector end 130 and a truncated end 132. When the connector 124 is inserted through the valve 118, it opens up the valve 118 and allows standard hospital equipment to be connected to the standard connector end 130 thereof. Alternatively, a valved connector, such as connector/adaptor 96 shown in FIG. 4, may also be connected to end 130 of connector 124.

In order to insert an endotracheal tube (ETT) into a patient using the device of FIG. 5 or FIG. 6, the following steps can be followed:

Insert the truncated tube 124 with a 15 mm universal connector in through the proximal end of a carefully selected laryngeal mask 110 (LM) fitted with a large valve to open up the valve for ventilation and secure the connector to the LM.

Thread the Bronchoscope through adapter 126 which has a smaller valve and a 15 mm fitting and further through the selected cuffed ETT through from its connector end to be readily available. At this stage, adaptor 126 is not connected to the LM.

Insert the LM and position into the patient and secure. Now we have a LM in situ with a large valve and a standard 15 mm connector on it. Now connect the ventilation device and ventilate the lungs of the patient.

Remove the truncated tube with the 15 mm connector 124 from the LM that is already positioned in the patient and quickly insert the ETT loaded with the Bronchoscope and the connector 125 and Adapter 126 through the large valve of the LM, connecting the ventilation device to Adapter 126 on to the connector 128 to ventilate and advance the ETT to the correct position visualising through the Bronchoscope. Once in place inflate the cuff of the ETT and secure the ETT to its correct position and continue to ventilate the patient's lungs.

When all is well and satisfactory, to remove the LM, disconnect the 15 mm connector 125 from the ETT, keeping the ETT in position, carefully remove the LM over the ETT as quickly as possible and insert the 15 mm connector 125 back on to the ETT. Check to see whether the position of the ETT is correct by listening to both lungs and secure the ETT before continuing to ventilate.

In some instances, during the removal of the LM over the correctly inserted ETT, there is a possibility of the ETT getting dislodged and coming off along with the LM as the length of the ETT may not be sufficiently long to hold on to by the operator during the removal of the LM. In this case, it is possible to insert another cuffed ETT with a smaller diameter through the outer end of the already installed ETT until the entire cuff of the second ETT has entered the lumen of the first ETT, followed by inflation of the cuff of the smaller ETT to securely fix it. Then the operator can have the grip and length to securely remove the LM.

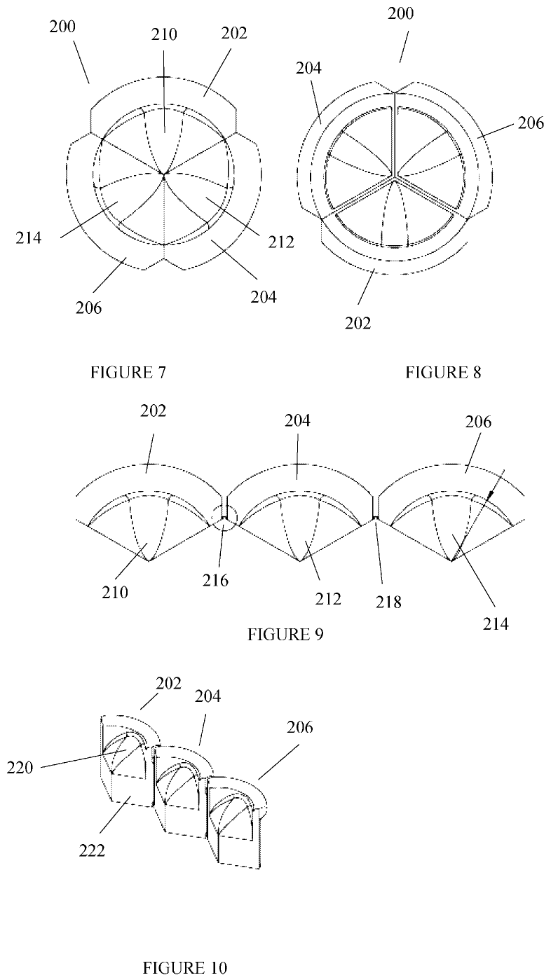

FIGS. 7 to 13 show various views of a valve that may be used in accordance with the present invention. Turning initially to FIGS. 7 and 8, the valve 200 comprises an upper peripheral portion made up of peripheral regions 202, 204, 206. The peripheral regions form the upper periphery of the valve (in this instance, the term “upper” is used to correspond to “proximal” or “upstream” parts of the valve and the term “lower” is used to refer to the “distal” or “downstream” parts of the valve). The upper periphery of the valve may be used to join the valve 200 to the inner periphery of the tube of the airway device. The valve 200 includes a first valve portion 210, a second valve portion 212 and a third valve portion 214. Each of the valve portions 210, 212, 214 circumscribes approximately one third of a circle, or about 120° of angle.

As shown in FIGS. 9 and 10, the valve portions 202, 204, 206 may be moulded together with the adjacent valve portions being connected through regions 216, 218. The valve portions can then be rotated relative to each other to form the shape shown in FIGS. 7 and 8 and the valve 200 can then be attached, such as by thermal welding or by an adhesive, to the inner wall of the tube of the airway device.

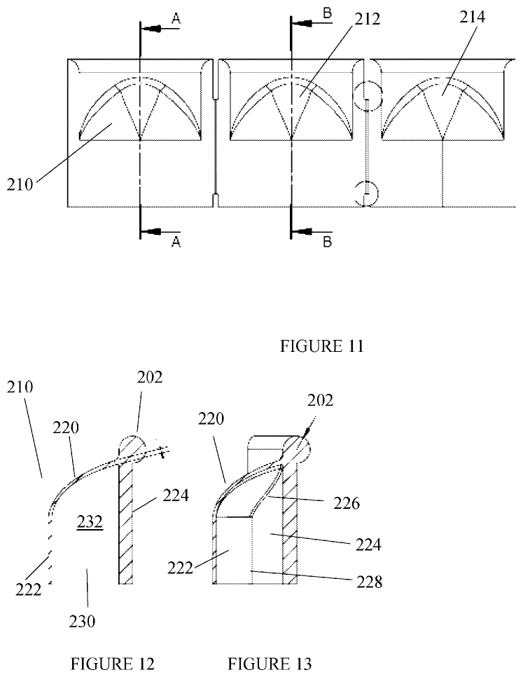

As shown in FIG. 10, valve portion 202 includes an upper surface 220 having a downwardly depending skirt 222 extending therefrom. As shown in FIG. 12, the upper surface 220 and downwardly depending skirt 222 are attached to an outer side wall 224. The outer periphery 202 can also be seen. As shown in FIG. 13, the upper surface 220 joins to the outer wall 224 along line 226 and the downwardly depending skirt 222 joins to the outer wall 224 along line 228.

As best shown in FIGS. 12 and 13, the downstream region 230 of the valve portion 210 is open and the upstream portion 232 is closed. In this manner, should gases flow from the downstream region 230 towards the upper region 232, the gas will enter the open downstream region 230 and fill the enclosed upper region 232 which will, in turn, force the downwardly depending skirt 222 into firm engagement with the adjacent skirts when in the closed position. If an endotracheal tube has been inserted through the valve 200, the endotracheal tube will spread apart the valve portions 210, 212, 214 but the walls of the downwardly depending skirts and part of the upper surfaces of the valve portions will closely fit against the endotracheal tube due to the flexibility and resilience of the skirts and surfaces. In this regard, the valve 200 may be made from a medical grade silicone or other suitable polymeric material that has both resiliency and flexibility. Further, should gases try to leak out through the valve 200 when an endotracheal tube has been inserted through the valve, the gas will enter the open downstream region 230 and the closed upstream region 232 and this will result in the gas effectively inflating the skirts and surfaces of the valve portions to bring them into closer conformity with the endotracheal tube. This also improves the seal around the endotracheal tube.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A device adapted for maintaining an airway in a patient, the device comprising a mask, the mask adapted to form a seal around the laryngeal inlet when properly inserted into a patient and an airway tube for providing ventilation gases and/or anaesthetic gases through the mask and to the lungs of the patient when the device is properly inserted in a patient, wherein the airway tube has a first proximal opening and a second proximal opening, wherein at least one of the first opening or the second opening is provided with a non-return valve, or both the first opening and the second opening are provided with a non-return valve, or a non-return valve is located upstream of a bifurcation point in at least one of the tubes located upstream of the bifurcated point, wherein the non-return valve comprises a plurality of valve portions, each valve portion having a surface extending inwardly from a periphery thereof, the valve portion having one edge that, in use, abuts an edge of an inwardly extending surface of an adjacent valve portion and another edge that, in use, abuts an edge of an inwardly extending surface of an adjacent valve portion when in the closed position, the surface having a skirt extending therefrom, wherein the surface and the skirt define a closed upstream region and an open downstream region and, in use, the edges of the surface and edges of the skirts of one valve portion abut on respective edges of the surface and edges of the skirt of an adjacent valve portion when in the closed position, wherein if gas tries to flow from the downstream end to the upstream end, the gas will flow into the open downstream region and act to push the valve portions into contact with each other.

2. A device as claimed in claim 1 wherein the airway tube comprises a bifurcated tube having a first proximal opening and a second proximal opening or the airway tube comprises a first tube having a second tube extending from a side thereof.

3. A device as claimed in claim 1 wherein the airway tube comprises a bifurcated tube and the airway tube has a distal portion and the airway tube comprises a bifurcated tube forming a proximal portion of the airway tube.

4. A device as claimed in claim 2 wherein the bifurcated tube is formed as a separate portion that is attached to a distal portion of the airway tube.

5. A device as claimed in claim 1 wherein the airway tube comprises a stem that connects to or is formed with the mask at a distal end thereof.

6. A device as claimed in claim 1 wherein the airway tube comprises a first tube and a second tube to form suction and vent channels in the device.

7. A device as claimed in claim 1 wherein the airway tube comprises a stem, a first airway tube and a second airway tube, the first airway tube and the second airway tube being in fluid communication with the stem.

8. A device as claimed in claim 1 wherein the first opening is connected to a source of ventilating gas and/or anaesthetic gas during use and the second opening provides access to the airway tube to enable an endotracheal tube to be inserted through the airway tube and into the larynx of a patient.

9. (canceled)

10. A device as claimed in claim 1 wherein the airway tube has a first tube and a second tube being in fluid communication with an airway tube stem, and the non-return valve is located in the first tube, or in the second tube, or the non-return valve is located in each of the first tube and the second tube.

11. A device as claimed in claim 1 wherein the non-return valve is located in the stem of the airway tube.

12. A device as claimed in claim 1 wherein the non-return valve is located at or near the first opening and the first opening is adapted to have an endotracheal tube or other instrument passed therethrough.

13. A device as claimed in claim 1 wherein the airway tube comprises an airway stem tube and a bifurcated connector is attached to the proximal end of the airway tube stem, the bifurcated connector having a first arm and a second arm, the non-return valve being located in the first arm, or in the second arm, or the non-return valve being located in each of the first arm and the second arm.

14-23. (canceled)

24. A device as claimed in claim 1 wherein the valve comprises three valve portions or four valve portions.

25. A method for positioning endotracheal tube into a patient, the method comprising establishing an airway in the patient using the device as claimed in claim 1, providing ventilation gas and/or anaesthetic gas to the patient through one of the first opening or second opening of the device, and inserting an endotracheal tube through the other of the first opening on the second opening whilst maintaining supply of the ventilation gas and/or the anaesthetic gas.

26. A method for positioning endotracheal tube into a patient, the method comprising:
establishing an airway in the patient using a device comprising a mask, the mask adapted to form a seal around the laryngeal inlet when properly inserted into a patient and an airway tube for providing ventilation gases and/or anaesthetic gases through the mask and to the lungs of the patient when the device is properly inserted in a patient, wherein the airway tube has a first proximal opening and a second proximal opening, wherein at least one of the first opening or the second opening is provided with a non-return valve, or both the first opening and the second opening are provided with a non-return valve, or a non-return valve is located upstream of a bifurcation point in at least one of the tubes located upstream of the bifurcated point, wherein the non-return valve comprises a plurality of valve portions, each valve portion having a surface extending inwardly from a periphery thereof, the valve portion having one edge that, in use, abuts an edge of an inwardly extending surface of an adjacent valve portion and another edge that, in use, abuts an edge of an inwardly extending surface of an adjacent valve portion when in the closed position, the surface having a skirt extending therefrom, wherein the surface and the skirt define a closed upstream region and an open downstream region and, in use, the edges of the surface and edges of the skirts of one valve portion abut on respective edges of the surface and edges of the skirt of an adjacent valve portion when in the closed position, wherein if gas tries to flow from the downstream end to the upstream end, the gas will flow into the open downstream region and act to push the valve portions into contact with each other;
providing ventilation gas and/or anaesthetic gas to the patient through one of the first opening or second opening of the device;
inserting an endotracheal tube through the other of the first opening on the second opening whilst maintaining supply of the ventilation gas and/or the anaesthetic gas.
